(19) United States
(12) Patent Application Publication
(10) Pub. No.: US 2025/0114582 A1
(43) Pub. Date: Apr. 10, 2025

(54) METHOD FOR EFFICIENT DRUG ADMINISTRATION ACROSS THE BLOOD-BRAIN BARRIER AND USES THEREOF

(71) Applicant: Carthera, Lyon (FR)

(72) Inventors: Michael Canney, Denver, CO (US); Guillaume Bouchoux, Villeurbanne (FR)

(21) Appl. No.: 18/907,240

(22) Filed: Oct. 4, 2024

Related U.S. Application Data
(60) Provisional application No. 63/542,856, filed on Oct. 6, 2023.

(51) Int. Cl. A61M 37/00; A61N 7/00

(52) U.S. Cl. CPC A61M 37/0092; A61N 7/00; A61N 2007/0021; A61N 2007/0073

(57) ABSTRACT

A method for delivering a therapeutic agent to a human brain by delivering systemically a therapeutic agent and applying low intensity pulsed ultrasound (“LIPU”) to the brain in the presence of circulating microbubbles. The therapeutic agent is delivered prior to applying LIPU if it has a plasma half-life of ≥1 h, and the therapeutic agent is delivered after applying LIPU if it has a plasma half-life of <1 h. In a second disclosed method, a first therapeutic agent is delivered systemically, LIPU is applied to the brain in the presence of circulating microbubbles, and a second therapeutic agent is delivered systemically. Also provided is a method for treating a brain disorder in a human by delivering a therapeutic agent systemically, administering a microbubble solution, and applying LIPU to the brain for a period of 1 min. to 30 min.

### RELATED APPLICATION

This application claims the benefit of U.S. Provisional Patent Application Ser. No. 63/542,856, filed on Oct. 6, 2023. The entire teachings of the above application are incorporated herein by reference.

### BACKGROUND

The blood-brain barrier (“BBB”) is a highly selective and protective barrier that separates the bloodstream from the brain and spinal cord. It is a critical component of the central nervous system (“CNS”) and plays a crucial role in maintaining a stable and controlled environment for the brain's proper functioning. The primary function of the BBB is to regulate the passage of substances (such as ions, molecules, and cells) between the blood and the brain tissue while preventing harmful or unwanted substances from entering the brain. However, the BBB may also prevent or delay the transport of substances that are potentially useful for treating brain diseases, such as chemotherapy drugs, antibodies, and hydrophobic molecules. Thus, the BBB is one of the largest obstacles to treating many brain diseases, including infections of the central nervous system, neurodegenerative diseases, and brain cancers, as the diseased brain tissue is unable to receive from the systemic circulation a therapeutically effective concentration of drug.

Previous attempts have been made to either temporarily disrupt the BBB or to bypass it for enhancing drug delivery to the brain. For example, mannitol has been used as an osmotic substance to increase BBB permeability for particular drugs such as methotrexate and carboplatin. However, the injection of mannitol is often associated with side-effects including hemorrhages, and the duration and magnitude of the induced BBB opening is difficult to control.

Convection enhanced delivery (“CED”) is another technique that has been explored to bypass the BBB. CED is performed by inserting a small catheter directly into the targeted brain region and then slowly infusing the drug directly into the tissue, thereby bypassing the BBB. Although CED has shown issues such as rapid drug elimination from tissue and limited regions of drug penetration from the infusing catheter, the technique continues to be explored in clinical trials (White et al. 2012, Contemp. Clin. Trials 33:320-331) but has not gained widespread clinical acceptance. It is also difficult to perform repeatedly and is typically used only for a single infusion of a substance.

Recently, a new technique has been proposed to temporarily disrupt the BBB and increase drug concentrations in the brain using low-intensity pulsed ultrasound (“LIPU”). When ultrasound (“US”) is applied to the brain in combination with systemic injection of micron-sized bubbles, i.e., microbubbles, the BBB permeability can be temporarily increased for a duration of up to several hours (Hynynen et al. 2001, Radiology 220:640-646; McDannold et al. 2008, Ultrasound Med Biol. 34:930-937; Park et al. 2012, J. Control. Release 162:134-142). This opening of the BBB has furthermore been shown to enhance the brain concentrations of systemically administered drugs (e.g., doxorubicin, BCNU, irinotecan, carboplatin, temozolomide, and trastuzumab) to therapeutic levels in pre-clinical studies (Treat et al. 2012, Ultrasound Med Biol. 38:1716-1725; Liu et al. 2010, Radiololgy 255:415-425; Wei et al. 2013, PLoS One 8: e58995; Beccaria et al. 2013, J. Neurosurg. 124:1602-1610; Park et al. 2012, J. Control. Release 162:134-142; Sonabend et al. 2023, Lancet Oncol., 24:509-522).

Accordingly, there is a need in the art for methods to repeatedly increase the concentration of systematically administered drugs in the brain in a more controlled and less invasive way.

### SUMMARY

To meet the need stated above, a method is disclosed for delivering a therapeutic agent to a human brain. The method is carried out by systemically delivering a therapeutic agent to a human and applying LIPU in the presence of circulating microbubbles to the brain for a period of 1 μs to 20 min. The LIPU results in opening of a blood-brain barrier volume of 1-1500 cm³. The therapeutic agent is delivered prior to applying the LIPU if the agent has a half-life in plasma of ≥1 h, or the therapeutic agent is delivered after applying the LIPU if the therapeutic agent has a half-life in plasma of <1 h.

A second method for delivering therapeutic agents to a human brain is provided. The method is accomplished by delivering systemically a first therapeutic agent to a human, applying LIPU to the brain in the presence of circulating microbubbles for a period of 1 μs to 20 min., and delivering systemically a second therapeutic agent. Like the method set forth above, the LIPU results in an opening of a blood-brain barrier volume of 1-1500 cm³. In this second method, the first therapeutic agent has a longer half-life in plasma than the second therapeutic agent, the first therapeutic agent is delivered prior to applying the LIPU, and the second therapeutic agent is delivered after applying the LIPU.

Finally, provided is a method for treating a brain disorder in a human. The method includes the steps of delivering a therapeutic agent systemically to a human suffering from a brain disorder, administering to the human a microbubble solution that circulates in the bloodstream, and applying LIPU to the brain of the human for a period of 1 min. to 30 min. The LIPU results in an opening of a blood-brain barrier volume of 1-1500 cm³ and the therapeutic agent is delivered by infusion starting at 15 to 90 min prior to applying the LIPU.

The details of one or more embodiments are set forth in the description below and in the appendices. Other features, objects, and advantages will be apparent from the detailed description, from the drawings, and also from the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The description below refers to the accompanying drawings, of which:

FIG. 1A is a plot of the permeability of the blood-brain barrier versus time after opening with ultrasound (time 0) in a two-compartment model, i.e., brain and plasma.

FIG. 1B is a plot of plasma concentration of carboplatin versus time showing the correspondence between the model (red curve) and that from the literature (Mulder et al. 1990, Br. J. Cancer 61:460-464).

FIG. 2A is a plot of predicted carboplatin concentration in the brain (relative to peak levels in plasma) versus time for the indicated infusion regimens, i.e., before application of ultrasound or after application of ultrasound (US).

FIG. 2B is a plot of normalized peak carboplatin concentration in the brain versus time of infusion start relative to ultrasound application, set at time 0.

FIG. 3 is a plot of plasma concentration of paclitaxel versus time showing the correspondence between the model (red curve) and that from the literature (Sparreboom et al. 2005, Clin. Cancer Res. 11:4136-4143).

FIG. 4A is a plot of predicted paclitaxel concentration in the brain (relative to peak levels in plasma) versus time for the indicated infusion regimens, i.e., before application of ultrasound or after application of ultrasound (US).

FIG. 4B is a plot of normalized peak paclitaxel concentration in the brain versus time of infusion start before or after to the time of ultrasound application, set at time 0.

FIG. 5A is a plot of normalized plasma drug concentration versus time for a hypothetical drug having an alpha half-life of 0.2 h that is infused immediately before (green curve) or after (red curve) ultrasound application. The estimated BBB opening is shown in a dashed curve.

FIG. 5B is a plot of brain drug concentration versus time for a hypothetical drug having an alpha half-life of 0.2 h that is infused immediately before (green curve) or after (red curve) ultrasound application.

FIG. 5C is a plot of normalized plasma drug concentration for a hypothetical drug having an alpha half-life of 0.2 h versus time relative to ultrasound application, set at time 0.

FIG. 5 D is a plot of normalized plasma drug concentration versus time for a hypothetical drug having an alpha half-life of 1.2 h that is infused immediately before (green curve) or after (red curve) ultrasound application. The estimated BBB opening is shown in a dashed curve.

FIG. 5E is a plot of brain drug concentration versus time for a hypothetical drug having an alpha half-life of 1.2 h that is infused immediately before (green curve) or after (red curve) ultrasound application.

FIG. 5F is a plot of normalized plasma drug concentration for a hypothetical drug having an alpha half-life of 1.2 h versus time relative to ultrasound application, set at time 0.

FIG. 6 shows a clinical trial for evaluating the safety and efficacy of transient disruption of the blood-brain barrier by low intensity pulsed ultrasound (“LIPU”) with an implantable ultrasound device.

FIG. 7A shows a diagram of the SonoCloud-9 System that consists of an implant containing nine 1-MHz, 10-mm diameter ultrasound emitters that are powered by a transdermal needle used to connect the device at each activation to an external generator that includes a touchscreen interface to guide the treatment and provide the energy to the implant.

FIG. 7B shows the activation procedure to disrupt the BBB that was performed monthly at the time of carboplatin infusion, with carboplatin infusion performed either immediately before (cohort D) or after (cohorts A, B, C) sonication.

FIG. 8 shows pre and post-sonication MRI images from two patients with nine emitters active showing the region of BBB disruption induced by the SonoCloud-9 System (red arrows indicate region of BBB disruption; blue line indicates position of SonoCloud-9 device).

FIG. 9A is a violin plot showing the percent T1 weighted MRI contrast enhancement of patients at different clinical sites. A significant difference in sonication-induced T1 contrast enhancement was found between sites due to MRI acquisition parameters and gadolinium contrast agent used, with Site 4 using Gadavist® and all other sites using Dotarem®.

FIG. 9B is a plot of T1 weighted MRI contrast enhancement from clinical site 1 versus time between the end of sonication and injection of gadolinium contrast agent. The time between sonication and gadolinium bolus at this site was 10-77 min. due to availability of MRI after sonication. There was a significant negative correlation between enhancement intensity and sonication to gadolinium injection time for treatments performed in site 1 (n=31 sonications, p=0.05), with an exponential decay fit indicating a half-closure time of 1.3 hours.

FIG. 10 shows successive T1 weighted MRI images from one patient taken pre-sonication once monthly for six months. This patient had an increase in T1 enhancement up to treatment Cycle 2 that then decreased over time with each monthly cycle of treatment. Overall, tumor growth was better controlled in the field of sonication (shown in green) than outside the field of sonication in patients treated in Cohort D that received carboplatin infusion prior to sonication to disrupt the BBB.

FIG. 11A is a plot of hyperintense T1 weighted MRI volume in the region of interest (ROI) versus days after tumor resection for patients in study cohorts C and D.

FIG. 11B shows tumor growth rate in mL/month for study cohorts C and D. Over the study duration, it was shown that tumor growth in cohort C (median=2.31 mL/month) was significantly higher than in cohort D (median=0.54 mL/month (Wilcoxon-Mann-Whitney test: p=0.04). When the region targeted by the implant was excluded from the analysis, there was no significant difference between the evolution of the T1 enhancement (p=0.55).

FIG. 11C is a visualization of the probability of enhancement in T1 weighted images at progression in which each of the circles depicts an emitter axis from an actual SonoCloud-9 implant in a patient.

FIG. 11D is a plot of enhanced T1 weighted signal at progression (% ROI volume) versus distance to the closest emitter axis. The percentage of ring-shaped ROIs surrounding emitter axes covered with hyperintense tumor at the end of the study were compared and was shown to be lower in Cohort D than Cohort C. The sonicated zone with BBB disruption corresponds to the 0-5 mm bin (10-mm cylinders), and effect on local tumor progression is observed up to 10-mm from the emitter axes (statistically significant up to 7.5 mm).

### DETAILED DESCRIPTION

As summarized above, a first method is disclosed for delivering a therapeutic agent to a human brain. In the method, the time period between completion of the step of delivering the therapeutic agent and the step of applying the LIPU is not more than 45 min. (e.g., not more than 0, 1, 2, 3, 4, 5, 10, 15, 20, 25, 30, 35, 40, and 45 min.).

In a particular method, if the therapeutic agent has a half-life in plasma of ≥1 h, the LIPU is applied within 0 to 30 min. (e.g., 0, 1, 2, 3, 4, 5, 10, 15, 20, 25, and 30 min.) after completion of delivery of the therapeutic agent.

In another particular method, if the therapeutic agent has a half-life in plasma of <1 h, delivery of the therapeutic agent commences within 0 to 30 min. (e.g., 0, 1, 2, 3, 4, 5, 10, 15, 20, 25, and 30 min.) after application of the LIPU.

As mentioned above, the LIPU is applied in the presence of circulating microbubbles. For example, LIPU is applied simultaneous with IV microbubble injection, for example Definity® microbubbles, at a dose of 10-100 μL/kg. Alternatively, Sono Vue microbubbles at a dose of 0.1 mL/kg can be introduced over a 30 s bolus infusion prior to or simultaneous with the start of LIPU application.

In any of the methods described, supra, the therapeutic agent is delivered over a period of time. For example, the therapeutic agent can be delivered by infusion over a period of 10 min. to 90 min. (e.g., 10, 20, 30, 40, 50, 60, 70, 80, and 90 min.) Preferably, the infusion time is not more than 60 min., more preferably not more than 30 min.

In any of the foregoing methods, the LIPU is applied to the brain at a pressure level of 0.5 MPa or higher, such as a pressure between 0.5 MPa and 2.00 MPa (0.5, 0.6, 0.7, 0.8, 0.9, 1.0, 1.1, 1.2, 1.3, 1.4, 1.5, 1.6, 1.7, 1.8, 1.9, and 2.00 MPa). These pressures represent a non-derated pressure as measured in H₂O for a given device system without accounting for attenuation by brain tissue. The pressure at the brain tissue is expected to be approximately 10% less than the above values for a sonication frequency of 1 MHz when the LIPU is applied in the absence of the skull bone and is dependent on the tissue path or resection cavity that is immediately in front of the transducer applying the ultrasound energy (Asquier, et al. 2019, J. Neurosurg. 132:875-883). Alternatively, the in situ pressure can be stated in terms of the mechanical index (“MI”). The MI here is defined as peak negative pressure (in MPa) divided by the square root of the frequency (in MHz) of the ultrasound wave. An MI of 0.1-2 can be used to induce blood-brain barrier opening with a given ultrasound transducer operating at either 1 MHz, or at another frequency in a range of 100 kHz to 10 MHz.

The LIPU in the above methods is applied (i) at a resonance frequency ranging from 0.5 to 1.5 MHz (e.g., 0.5, 0.75, 1.0, and 1.5 MHz) and (ii) in pulses of duration about 25 ms and a pulse repetition frequency of about 0.5 Hz to 1 Hz. The LIPU in these methods can have a mechanical index from 0.1 to 2.00.

LIPU can be performed as described in U.S. Pat. No. 10,981,021.

Preferably, the LIPU in the above methods is applied by one or several ultrasound transducers implanted in a burr hole or a bone flap in the skull of the human. The LIPU can be applied by one or several discrete implanted ultrasound transducers, or by one or several compact transducer arrays. For example, the array can include 9 discrete 1 cm diameter 1 MHz ultrasound transducers arranged in a 3×3 grid with a 20-mm pitch. Such a device is described in WO2016/097867. The ultrasound transducers can be activated sequentially to apply LIPU in any of the preceding methods.

As an alternative, LIPU can be applied to the brain through the skull as described in U.S. Pat. Nos. 10,166,379 or 7,674,229.

As mentioned above, the disclosed method is for delivering a therapeutic agent to the brain. The therapeutic agent can be, but is not limited to (i) a drug typically used to treat brain cancer such as carboplatin, temozolomide, lomustine, carmustine, irinotecan, topotecan, trastuzumab, doxorubicin, and panobinostat; (ii) immunotherapies such as therapeutic antibodies targeting PD-1 or CTLA-4, a CAR T cell, an NK cell, and (iii) neurodegenerative disease treatments such as anti-amyloid therapies (e.g. lecanumab or aducanumab), anti-tau therapies, or anti-alpha-synuclein therapies. Any of these therapeutic agents can be delivered by infusion over a period not exceeding 60 min. (e.g., 30-60 min.) in the above-described methods.

In a particular method, two distinct therapeutic agents are delivered to the brain. The two distinct agents can be selected from those described above. The two therapeutic agents can be delivered simultaneously, or one can be delivered prior to delivering the other. In an exemplary method, the two distinct therapeutic agents are carboplatin and doxorubicin. In another method, the two distinct therapeutic agents are an anti-PD1 therapeutic antibody and doxorubicin.

A second method for delivering therapeutic agents to a human brain is also provided. In this specific method, (i) a first therapeutic agent is delivered to the human, e.g., by infusion, (ii) LIPU is applied to the brain in the presence of an ultrasound contrast agent for a period of 25 ms to 20 min., and (iii) a second therapeutic agent is delivered systemically. In this second method, the first therapeutic agent has a longer half-life in plasma than the second therapeutic agent, the first therapeutic agent is delivered prior to applying the LIPU, and the second therapeutic agent is delivered after applying the LIPU. Like the first method set forth above, the LIPU results in an opening of a blood-brain barrier volume of 1-1500 cm³.

Finally, provided is a method for treating a brain disorder in a human by delivering a therapeutic agent systemically to the human, administering to the human an ultrasound contrast agent, and applying LIPU to the brain of the human for a period of 1 min. to 30 min. Again, the LIPU results in an opening of a blood-brain barrier volume of 1-1500 cm³. In this method, the therapeutic agent is delivered by infusion starting at 15 to 90 min. prior to applying the LIPU.

The brain disorders that can be treated by this method include, but are not limited to, cancer, Parkinson's disease, Amyotrophic lateral sclerosis, Alzheimer's disease, Huntington disease, and frontotemporal dementia. The cancer can be a glioma, e.g., astrocytoma, glioblastoma, oligodendroglioma, and ependymoma, or secondary brain metastases that originated from the lung, breast, skin, ovaries, or intestines.

In a specific method, the brain disorder is cancer and the therapeutic agent is carboplatin delivered at a dose of AUC3-7. In this method, it is preferred that the LIPU is applied for 3 to 5 min., e.g., 3, 3.5, 4, 4.5, and 5 min.

In the method set out in the three preceding paragraphs, the LIPU is applied by an ultrasound transducer implanted in a burr hole or bone flap in a skull of the human. Similar to the first method, the LIPU can be applied by an array of implanted ultrasound transducers, e.g., a 3×3 grid with a 20-mm pitch of 9 discrete 1-cm diameter, 1-MHz ultrasound transducers. Again, the ultrasound transducers can be activated sequentially to apply LIPU in any of the preceding methods.

In a particular method, the therapeutic agent is infused through a catheter. A more specific method includes the steps of (i) infusing the therapeutic agent through the catheter for a period less than 60 min., (ii) flushing the catheter with saline for a period of 20 s to 10 min, (iii) preparing the microbubble solution, (iv) connecting the ultrasound transducer electrically to a radio frequency generator, and (v) injecting the microbubble solution into the catheter. Of note, steps (iii) and (iv) can be performed concomitantly with steps (i) and (ii) to permit application of the LIPU immediately after completion of step (v).

In one method, the therapeutic agent, e.g., carboplatin, is delivered by infusion for 30 min. through the catheter prior to the flushing step and the LIPU is applied for a period of 4.5 min after injection of the ultrasound contrast agent.

In any of the above methods, it is preferable that the method is completed within 1 hour or less, including drug infusion time and time of application of LIPU.

Optionally, the methods above include obtaining a magnetic resonance image of the brain of the human to determine the extent of blood-brain barrier volume opening.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever. All publications and patent documents cited herein are incorporated by reference in their entirety.

### EXAMPLES

### Example 1: Simulation Studies

### Carboplatin

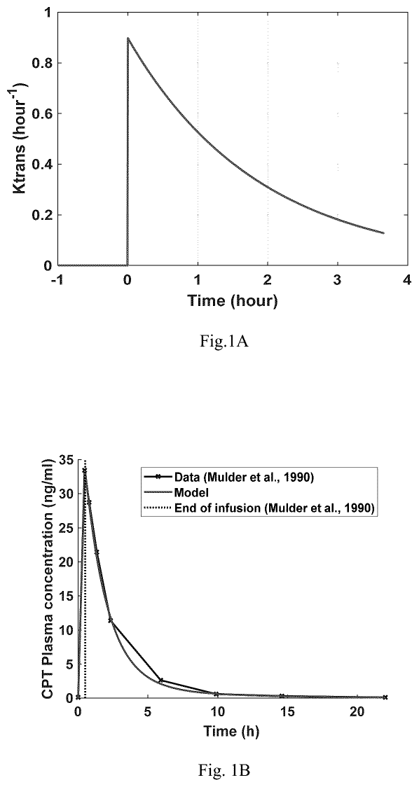

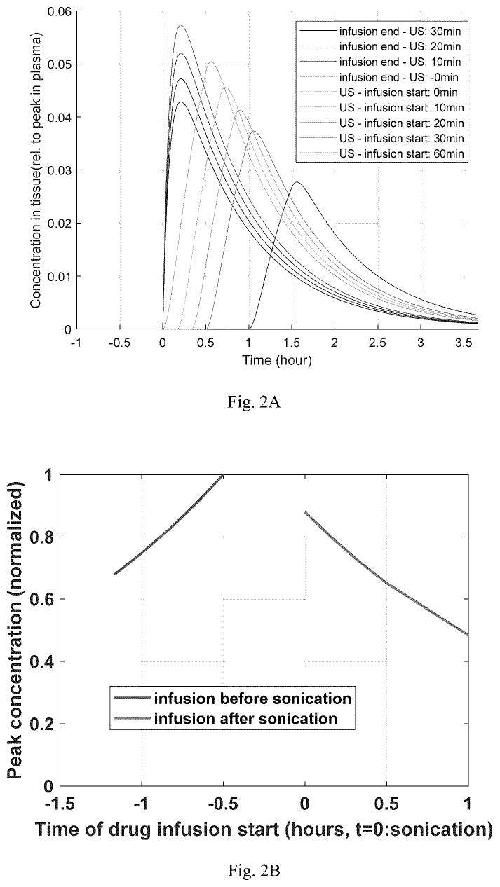

A 2-compartment model was constructed assuming a 1.3 h BBB closure half-time (see FIG. 1A) and using a typical carboplatin plasma kinetics from the literature (alpha half-life˜1 h, beta half-life˜6 h; see FIG. 1B). The model was run to simulate peak carboplatin concentration in brain parenchyma, considering a 30-minute infusion and a <5 min sonication. The results are shown in FIGS. 2A and 2B.

The simulations showed that starting the sonication immediately after the end of infusion allows for a +10% carboplatin concentration in the brain compared to starting the infusion immediately after the end of sonication. See FIG. 2A.

Considering a sonication before carboplatin infusion: a 30-minute delay between the end of the sonication and the start of carboplatin infusions causes a 25% decrease in carboplatin concentration in the brain. See FIG. 2B

Considering a sonication after carboplatin infusion: a 30-minute delay between the end of the carboplatin infusion and the start of sonication causes a 25% decrease in carboplatin concentration in the brain. See FIG. 2B.

### Paclitaxel

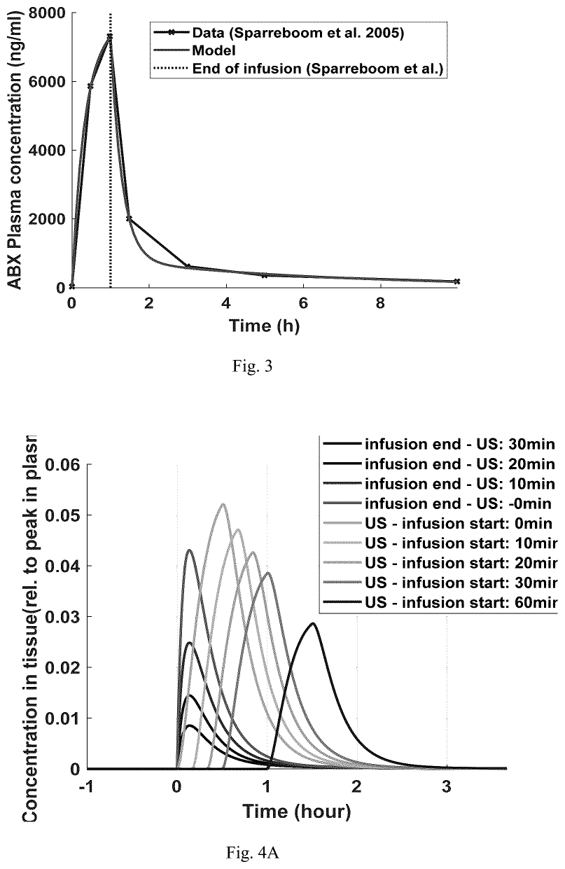

A 2-compartment model was constructed assuming a 1.3 h BBB closure half-time (see FIG. 1A) and using a typical paclitaxel plasma kinetics from the literature (alpha half-life˜0.5 h, beta half-life several hours: see FIG. 3). The model was run to simulate peak paclitaxel concentration in brain parenchyma, considering a 30-minute infusion and a <5 min sonication. The results are shown in FIGS. 4A and 4B.

According to these simulations, starting the infusion of paclitaxel immediately after the end of sonication allows for a ˜+20% paclitaxel concentration in the brain compared to starting sonication immediately after the end of infusion. See FIG. 4A.

Considering a sonication before paclitaxel infusion: a 30-minute delay between the end of the sonication and the start of carboplatin infusions causes a 26% decrease in paclitaxel concentration in the brain. See FIG. 4B.

Considering a sonication after paclitaxel infusion: a 30-minute delay between the end of the paclitaxel infusion and the start of sonication causes a >70% decrease in paclitaxel concentration in the brain. See FIG. 4B.

### General Model

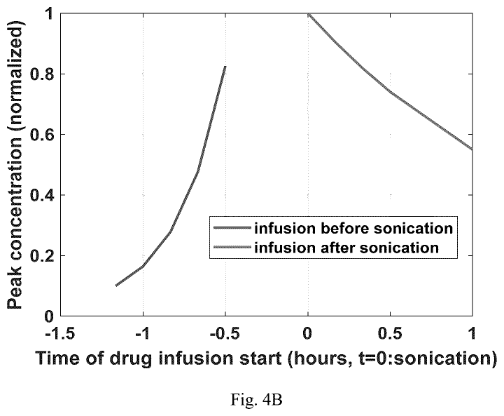

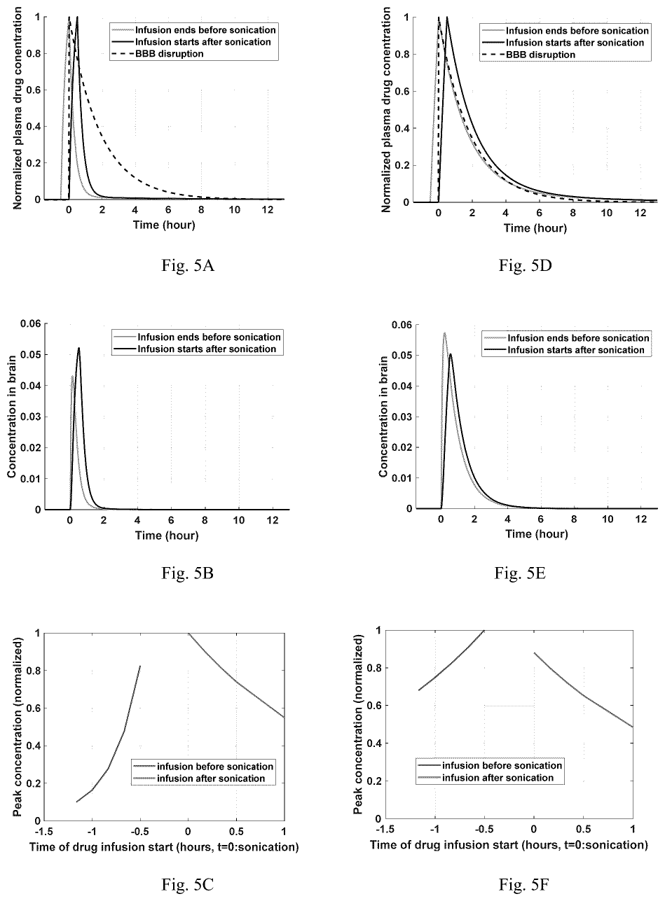

Simulations of drug concentration in the brain were carried out assuming that a drug with an alpha half-life of 0.2 h or a drug with and alpha half-life of 1.2 h is infused (30 min. infusion time) either before or after ultrasound. The results are shown in FIGS. 5A-5F. Normalized concentration of drug in plasma, BBB disruption, and drug concentration in the brain for scenarios with no delay between end of infusion and sonication or sonication and start of infusion of a drug with an alpha half-life of 0.2 h are shown in FIGS. 5A and 5B. Similar simulation data is shown in FIGS. 5D and 5E for a drug with an alpha half-life of 1.2 h. The peak drug concentrations in the brain for scenarios with additional delays between infusion and sonication are shown in FIGS. 5C (alpha half-life 0.2 h) and 5F (alpha half-life of 1.2 h.).

The above simulations showed that:
(i) It is critical to minimize time between start of sonication and end of infusion, or end of sonication and start of infusion. A 30-minute delay causes a-25% decrease in drug concentration in the brain.
(ii) A long sonication procedure based on scanning the ultrasound beam (>20 minutes) could lead to a heterogeneous drug distribution in the targeted volume (variability on the order of 25% for a 30-minute sonication procedure)
(iii) Drugs with a shorter (<1 h) alpha half-life (distribution phase half-life) such as paclitaxel should preferentially be infused after sonication (doing the opposite could lead to a ˜20% decrease in drug concentration level in brain), and
(iv) Drugs with a longer half-life (>=1 h) such as carboplatin should preferentially be infused before ultrasound.

According to these simulations, beta half-life (elimination phase) has a limited impact on drug concentration in the brain.

### Example 2: Clinical Trial

### Study Design

A prospective, open-label, multi-center, single-arm, dose escalation, phase 1/2a clinical trial was conducted enrolling recurrent Glioblastoma Multiforme (“GBM”) patients at four clinical sites in France and two sites in the United States. All patients provided written informed consent in accordance with institutional guidelines. Approval was obtained from the ANSM (French National Health Agency) and from the US FDA (Food and Drug Administration). The study was conducted in accordance with good clinical practices. The study began in February 2019 and follow-up was completed in November 2022.

### Patient Selection

Patients experiencing recurrence (any) of a histologically proven primary GBM, after at least a first-line standard of care (radiation with concurrent and adjuvant temozolomide) were recruited. Qualifying patients with good performance status (Karnofsky Performance Status ≥70), were eligible for carboplatin-based chemotherapy and tumor resection, with tumor size limited to 70 mm in diameter on T1w contrast-enhanced Magnetic Resonance Imaging (“MRI”). Patients receiving steroids, should be stable and have received less than 40 mg prednisone dose (dexamethasone ≤6 mg) for at least 7 days preceding study participation.

### Trial Design

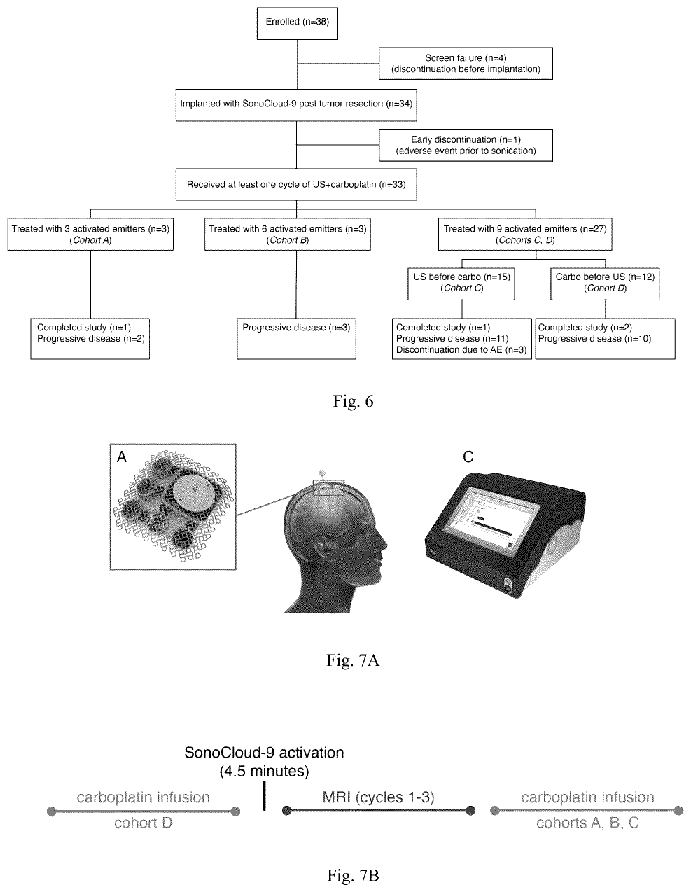

The trial was designed to evaluate the safety and efficacy of transient disruption of the blood-brain barrier by low intensity pulsed ultrasound (“LIPU”) with an implantable ultrasound device, i.e., the SonoCloud-9 implantable device (“SC9”; Carthera, Lyon, France). The trial first evaluated the Dose Limiting Toxicity (“DLT”) of escalating numbers of ultrasound beams (3, 6, and 9 beams) at constant acoustic pressure (1.03 MPa) using a 3+3 dose escalation design (cohorts A, B and C) and then confirmed the safety and efficacy of BBB opening in two expansion cohorts (cohorts C & D). See FIG. 6.

For the Phase 1 (escalation of the number of active emitters/beams), the main objective was to identify the Maximum Tolerated Dose (“MTD”) defined as the highest active-beams level at which ≤1 DLT occurred in a maximum of 6 patients by cohort. The DLT was defined as any Common Terminology Criteria for Adverse Events (CTCAE), version 5.0, Grade 3 or higher, event at least possibly attributable to the sonication or to the sonication plus carboplatin procedure that occurred within 15 days and that did not respond to optimal medical management (including steroids) within 7 days, including symptomatic intracranial hemorrhage of any grade, seizure of grade 3 or 4 (status epilepticus) regardless of time of resolution or symptomatic stroke of any grade. The Maximum Tolerated Dose (“MTD”) was defined as the highest active-beams level at which ≤1 DLT occurs in a maximum of 6 patients by cohort.

In Cohort A, SC9 at the 3 active beams level was given to 3 patients. In Cohort B, SC9 at the 6 active beams level was given to 3 new patients. In the absence of DLT, SC9 at the 9 active beams level was given to 3 new patients (cohort C). If 0 of the 3 patients experienced a DLT, then the 9 active beams level was determined to be the MTD and those patients of groups C of the phase 1 study were added to the expansion phase 2a patients treated at MTD (groups C, and D), to assess the blood brain barrier opening at the maximum number of SonoCloud-9 emitters tolerated. An Independent Data Safety Monitoring Board evaluated safety data and advised continuation of the trial after each cohort of the dose escalation portion.

In the extension Phase 2a, BBB opening efficacy was evaluated as the percentage of successful ultrasound sessions. A successful ultrasound session was defined by the number of emitters for which the BBB opening was Grade 2 (sub arachnoid and grey matter contrast enhancement) or Grade 3 (sub arachnoid, grey & white matter contrast enhancement) during the first three cycles by comparison of pre- and post-LIPU session T1w magnetic resonance imaging (MRI) as defined previously. See Carpentier et al., 2016 Sci. Transl. Med. 8: 343re2 and Asquier et al. 2019, J. Neurosurg. 132:875-883.

The secondary endpoints included the frequency and severity of adverse events (incidence of adverse events summarized by system organ class and/or preferred term and severity) based on the CTCAE, version 5.0, the time to and localization of recurrence(s) on magnetic resonance imaging, feasibility of the procedure considering the time required for the sonication (from beginning of needle connection to the end of ultrasound emission and needle extraction) at the first 3 sonications of each patient, and the total time for SonoCloud 9 positioning using the SonoCloud 9 template. Six-month progression-free survival (6m-PFS), median progression-free survival (mPFS), and 1-year overall survival (1y-OS) and median OS (mOS) were also evaluated. In this trial, time to progression or to death was calculated from time of surgery/device implantation to time of events.

For each patient, participation was planned to last approximately 7.5 months: from inclusion to surgery was a maximum of 14 days, from surgery to first sonication (Cycle 1) was a minimum of 9 days to a maximum of 14 days to allow for surgery recovery. Patients were then treated until month 6, tumor progression, or premature discontinuation, whichever came first. The number of cycles was defined by the chemotherapy frequency (every 4 weeks). The outcome was documented at the end of study visit that took place within 1 month of the event. Patients who did not progress by month 6 were allowed to continue ultrasound treatment (sonication with chemotherapy) as initiated, if considered to be in the best interests of the patient by the Investigator.

### Patient Eligibility

Patients eligible for tumor resection surgery and carboplatin chemotherapy with histologically proven recurrent de novo glioblastoma after at least a first-line standard of care (maximal safe resection, if feasible, radiation with temozolomide, then maintenance temozolomide) were proposed to participate in the trial. The pre surgery tumor size was limited to a maximum diameter of 70 mm. Patients with multi-focal and posterior fossa tumors were not eligible. Patients with Karnofsky performance status <70, at risk of surgery site infection and patients who had undergone antiangiogenic treatment or patients in need of continuous antiplatelet therapy were also excluded. The dose of steroids was limited to 40 mg of prednisone (or dexamethasone ≤6 mg) for at least 7 days, at inclusion. The use of non-absorbable hemostatic agent or dura matter substitutes were not authorized at surgery.

### Device Implantation

The implantation of the SonoCloud-9 device, shown in FIG. 7A, was performed by a trained neurosurgeon at the end of planned standard tumor resection. The SC9 implant is designed to replace a 58 mm×58 mm bone flap that is removed during surgical resection. At the beginning of the surgery, after skin opening, the surgeons positioned a template using a neuro-navigation pointer to ensure that the implant would cover the maximum infiltrative region surrounding the tumor resection bed (high-signal FLAIR region). Once the SC9 implant location was set, the template was used to trace the craniectomy size on the skull of the patient for the implant location. The surgeons performed a standard craniotomy, opened the dura mater, and performed the tumor debulking/resection as per the routine. Then the dura matter was closed, the SC9 implant was then secured on a window in the skull epidurally and recovered by the skin.

### Sonication Procedure

The SC9 device contains no internal energy source and is activated on demand by connecting the device to an external generator using a transdermal needle, as shown in FIG. 7A. Each sonication step consists of the generation of sequential pulses from each emitter (1 MHz, 25 ms pulse, 0.5 Hz, 270 seconds) in combination with the IV administration of the ultrasound resonator (microbubbles [MB], Definity®/Luminity® 10 μL/kg, Lantheus, N. Billerica, MA). The ultrasound resonator is injected as a 30-second bolus at the start of the ultrasound sonication procedure. Sonication was performed prior (within 60 minutes) to the carboplatin chemotherapy at cycles 1, 2 and 3 and no more than 30 minutes before the start of carboplatin therapy for the following cycles, for cohorts A, B, and C. For cohort D, the sonication was performed immediately after the completion of the carboplatin infusion. An illustration of the sequence of carboplatin infusion/device activation is shown in FIG. 7B. After completion of the six sonication cycles specified in the protocol or in the case of progression/premature discontinuation of the trial and prior to the end of study visit, the removal of the device could be performed unless considered as contra-indicated by the investigator or refused by the patient.

### Carboplatin Administration

Standard carboplatin chemotherapy using any FDA/ANSM-approved, therapeutically equivalent carboplatin injectable drug product, with a target AUC of 4-6 mg/ml*min (AUC as per local practice and investigators judgement) was given according to the Calvert formula (Calvert et al., 1989, J. Clin. Oncol. 7:1748-1756). Cycles were to be repeated every 4 weeks provided the absolute neutrophil count had recovered to ≥1500 cells/mm³ and the platelet count was at least 100,000 cells/mm³. Subsequent dosages were adjusted for toxicity as needed per local practice.

### Study Assessment

All patients were clinically assessed at least once a month (prior to the next cycle) and included standard laboratory blood analyses (complete blood counts, chemistry with liver and kidney function tests), and also a contrast-enhanced MRI within 2 days prior to sonication. Patients discontinued the study if tumor progression was identified according to the Response Assessment in Neuro-Oncology criteria (Wen et al., 2010, J. Clin. Oncol. 28:1963-1972). For patients continuing the study, subsequent MRI exam was to be performed immediately after LIPU/MB treatment.

A 3.0T MRI was used for all imaging exams at each site. At each exam, standard FLAIR, T1-weighted contrast-enhanced, SWAN, and diffusion sequences were obtained. T1-weighted MR images were analyzed to grade the type and extent of BBB disruption for post-sonication images and for tumor evolution in pre-sonication images. The gadolinium agent used was dependent on the site and the following macrocyclic agents were allowed on protocol and were administered according to their respective labels: DOTAREM® (gadoterate meglumine), GADAVIST® (gadubutrol), or PROHANCE® (gadoteridol). In order to limit the exposure to gadolinium agents, post-MRI procedures were performed only at Cycles 1, 2, and 3.

### BBB Opening Assessment on MRI

The effectiveness of BBB opening with the SC9 was assessed centrally by comparison of gadolinium enhanced MR images acquired before and after ultrasound sessions. The analysis was performed using the automated image processing pipeline according to the algorithm previously published (Asquier, et al. 2019, J. Neurosurg. 132:875-883). In this grading analysis, emitters in front of the resection cavity and of residual hyperintense tumor were excluded. Relative gadolinium enhancement maps from pre- to post-sonication images were computed after bias correction, brain segmentation (Ashburner et al., 2005, Neuroimage 26:839-851), normalization and non-rigid registration (Yushkevich et al., Neuroinformatics 17:83-102). Sonicated regions of interest (ROI) were defined by 10-mm diameter×75-mm length cylinders in front of each of the 9 emitters of the implant, considering only brain tissue that was not enhanced prior to sonication. The volume with detectable ultrasound-induced gadolinium enhancement was determined in the sonicated ROI by thresholding the enhancement map (threshold level: 1st centile of non-sonicated control ROI). A BBB disruption grade was automatically assigned to each emitter with enhanced volume >0.5 mL using the 0-3 scale defined in (Carpentier et al., 2016, Sci. Transl. Med. 8:343re2-343re2): grade 0-1 for enhancement in the subarachnoid space; otherwise, grade 2-3 was assigned (enhancement in grey or/and white matter). Additionally, as a metric of ultrasound-induced enhancement intensity, the 90th centile of relative enhancement in the sonicated ROI was calculated.

### Contrast Enhancing Tumor Progression Assessment on MRI

To evaluate the local effect of the treatment, an analysis was performed using the monthly pre-ultrasound and end-of-study MR images. The T1w contrast-enhancing tumor-related region segmented with a semi-automatic method (ITK Snap) (Yushkevich et al. 2006, Neuroimage 31:1116-1128). The total volume of this hyperintense region was evaluated at each treatment cycle in the whole brain, and in the region targeted by the implant, considering nine 20×80 mm cylinders in front of the emitters. This region corresponded to 10×75 mm cylinders with an additional diffusion margin of 5 mm (Goldwirt et al., 2016, Cancer Chemother. Pharmacol. 77:211-216).

To further evaluate tumor progression likelihood as a function of distance to the region targeted by the emitters, the difference of this hyperintense volume between the last MRI and the first cycle was used as a progression mask. The distribution of the volume of this progression mask relative to the distance to the axes of the nine emitters was computed.

### Statistical Analysis

A BBB opening success was defined by a T1W contrast enhancement in grey matter (grade 2) or in grey and white matter (grade 3) on two-third of the emitters. The proportion (x) of ultrasound sessions that were classified as being successful in opening the BBB was compared to an objective performance criterion (OPC) of 0.30 at the significance level of a 1-sided 2.5%. The BBB opening effectiveness was demonstrated if the lower limit of the 95% CI is higher than 0.30. Descriptive statistics were used as applicable to summarize the study data unless otherwise specified.

### Results

### Patient Profiles

Between February 2019 and June 2021, a total of 38 patients were registered for the trial. There were four patients who signed consent and subsequently did not continue the study due to screen failures (meningitis, tumor progression, COVID infection, low platelet counts). Thirty-four (34) patients underwent tumor resection and implantation of the SonoCloud-9 device, and 33 patients underwent at least one sonication procedure associated with carboplatin chemotherapy (FIG. 6). The number of activated emitters was escalated in cohorts of 3 patients from cohort A: 3 emitters, cohort B: 6 emitters and cohort C+D: 9 emitters for ultimately a total of 27 patients treated with all 9 ultrasound emitters activated.

Details of patient characteristics are summarized below in Table 1.

TABLE 1
Patient Characteristics.

| | All Cohorts | Cohort C | Cohort D |
|---|---|---|---|
| Demographics | (N = 34) | N = 15 | N = 12 |
| Sex | | | |
| Male | 19 | 7 | 7 |
| Female | 15 | 8 | 5 |
| Age (years) | | | |
| Mean (SD) | 56.4 (10.6) | 58.4 (10.0) | 54.3 (12.1) |
| Median | 58.0 | 59.0 | 55.0 |
| Time since initial diagnosis | | | |
| (months) to enrollment | | | |
| Mean (SD) | 21.5 (21.0) | 22.0 (26.0) | 17.2 (14.4) |
| Median | 13.2 | 11.7 | 11.2 |
| Recurrence | | | |
| 1 | 32 | 15 | 12 |
| 2 | 1 | 0 | 0 |
| 4 | 1 | 0 | 0 |
| Tumor Diameter, Max (mm) | | | |
| Mean (SD) | 30 (13.5) | 30 (14) | 34 (12) |
| MGMT status | | | |
| Methylated | 17 | 10 | 4 |
| Unmethylated | 17 | 5 | 8 |
| IDH wild-type | | | |
| Yes | 33 | 14 | 12 |
| No | 1 | 1 | 0 |
| KPS | | | |
| 100 | 2 | 1 | 1 |
| 90 | 20 | 9 | 7 |
| 80 | 7 | 2 | 3 |
| 70 | 5 | 3 | 1 |
| Steroid pre-Surgery | | | |
| Yes | 10 | 5 | 4 |
| No | 24 | 10 | 8 |
| Steroid pre-Cycle 1 | | | |
| Yes | 21 | 9 | 9 |
| No | 12 | 6 | 3 |
| Treatment Delivery | | | |
| Device Implantation | 34^{¶} | 15 | 12 |
| Sonication and carboplatin | 90 + 11^{†} | | |
| cycles | | | |
| Carboplatin Administration | | | |
| Time sonication to | | 63.7 [±9.9] | |
| carboplatin [min] | | | |
| Time end of carboplatin − | NA | NA | 13.8 (±6.5) |
| sonication [min] | | | |

^{¶}one patient died 7 days after surgery secondary to pulmonary embolism
^{†}3 patients continued treatment beyond the protocol prescribed 6 cycles at the discretion of the local investigators for a total of 11 additional cycles

Patients (56% male) had a median age of 58 years and all except two patients were treated at first recurrence (94%). Two tumors harbored an IDH mutation (6%). Half of the tumors were MGMT methylated. Patient characteristics of cohort C (sonication first and delay to start of carboplatin) and cohort D (sonication immediately preceding carboplatin) were comparable except for MGMT methylation favoring cohort C (66% methylated vs 33% in cohort D, respectively).

### Treatment Characteristics

The total time for the resection surgery including SonoCloud-9 implantation was a mean [SD] of 3.49 [±1.95] hours. A total of 90 sonications were performed per protocol (up to 6 cycles), with an additional 11 sonications performed off protocol (expansion for patients deemed benefitting from the treatment) in 3 patients who were not progressing after 6 cycles. Fifty-six percent (56%) of patients received ≥2 cycles of ultrasound with carboplatin. The mean overall duration of the sonication procedure was 9.9 minutes [SD: ±3.38 min]. In cohort D (n=12 patients), the mean time from the end of carboplatin infusion to sonication was 13.8 minutes [SD: ±6.5] minutes, while in Cohort C (n=15 patients), the mean time from the end of sonication to the start of carboplatin infusion was 63.7 minutes [SD: 9.9], with a mean time to the end of carboplatin infusion of 120 minutes.

Dose-Limiting Toxicity, Tolerability, and Safety of Carboplatin Delivered with Concomitant LIPU/MB-Based BBB Opening

All patients implanted with the SonoCloud-9 were included in the safety analysis, as shown below in Table 2.

TABLE 2
Summary of treatment emergent adverse events (TEAEs) for all grade and grade
3 related to the investigational procedure, according to the common terminology criteria
for adverse events (CTCAE).

| | | CTCAE |
|---|---|---|
| | CTCAE all grade | Grade 3 |
| System Organ Class/Preferred Term | (N = 34) | (N = 34) |
| Patients with Any Related TEAEs (Overall) | 33 (97%) | 12 (35%) |
| Nervous system disorders | 25 (74%) | 3 (9%) |
| Dizziness | 8 (24%) | |
| Headache | 7 (21%) | |
| Diplopia/Vision blurred | 6 (17%) | |
| Aphasia | 4 (12%) | |
| Meningocele acquired | 3 (9%) | |
| Paraesthesia | 3 (9%) | |
| Cerebrospinal fluid leakage | 2 (6%) | |
| Dysarthria | 2 (6%) | |
| Presyncope | 2 (6%) | 2 (6%) |
| Seizure | 2 (6%) | |
| Balance disorder | 1 (3%) | |
| Confusional state | 1 (3%) | |
| Hypoaesthesia | 1 (3%) | |
| Lacunar stroke | 1 (3%) | |
| Language disorder | 1 (3%) | |
| Monoparesis | 1 (3%) | |
| Skin and subcutaneous tissue disorders | 19 (56%) | 6 (18%) |
| Pain of skin | 19 (56%) | 6 (18%) |
| General disorders and administration site conditions | 8 (24%) | 1 (3%) |
| Fatigue/Asthenia | 6 (18%) | 1 (3%) |
| Implant site pain | 1 (3%) | |
| Injury, poisoning and procedural complications | 3 (9%) | |
| Post procedural oedema | 1 (3%) | |
| Scar | 1 (3%) | |
| Subarachnoid haemorrhage | 1 (3%) | |
| Ear and labyrinth disorders | 2 (6%) | |
| Vertigo | 2 (6%) | |
| Infections and infestations | 2 (6%) | 2 (6%) |
| Wound infection/Postoperative wound infection | 2 (6%) | 2 (6%) |
| Psychiatric disorders | 2 (6%) | |
| Anxiety | 1 (3%) | |
| Depression | 1 (3%) | |

The device implantation and repeated BBB opening procedure were well-tolerated in all patients. No DLTs were observed and all nine ultrasound emitters were safely activated in all patients of cohorts C and D. Higher grade possibly or likely device-related adverse events were grade 3 pre syncope in 2 patients, and grade 3 fatigue in one patient each. Wound infections (grade 3) following resection surgery and implantation of the device were also reported in two patients, which resolved after antibiotic treatment but led to treatment discontinuation after cycle 1 for one of the patients. Six patients (18%) complained of transient, yet severe pain (Grade 3) upon connection of the device with the needle connection procedure. No grade 4 events were recorded. One fatal event (pulmonary embolism) occurred 7 days post-surgery, but this patient did not receive any sonications/activation of the device due to the event happening prior to initiating cycle 1. This event was considered to be related to underlying conditions and not to the implantation procedure.

Lower grade treatment emergent adverse events (TEAEs) reported during sonication and included pain in the scalp, nausea, dizziness, headache, transient aphasia and blurred vision, as shown in Table 2. These presumed focal deficit TEAEs resolved within 15 minutes after the sonication procedure, except for one patient who reported blurred vision (Grade 1) that lasted for three months. None of these low-grade TEAEs prevented patients from receiving additional cycles of sonication and carboplatin administration.

The pattern of hematological and non-hematological toxicities secondary to carboplatin (e.g. bone marrow suppression, anemia, and vomiting) was as expected. Neutropenia led to discontinuation of treatment after cycle 1 for two patients (6%) not recovering within the delay specified between cycles in the protocol.

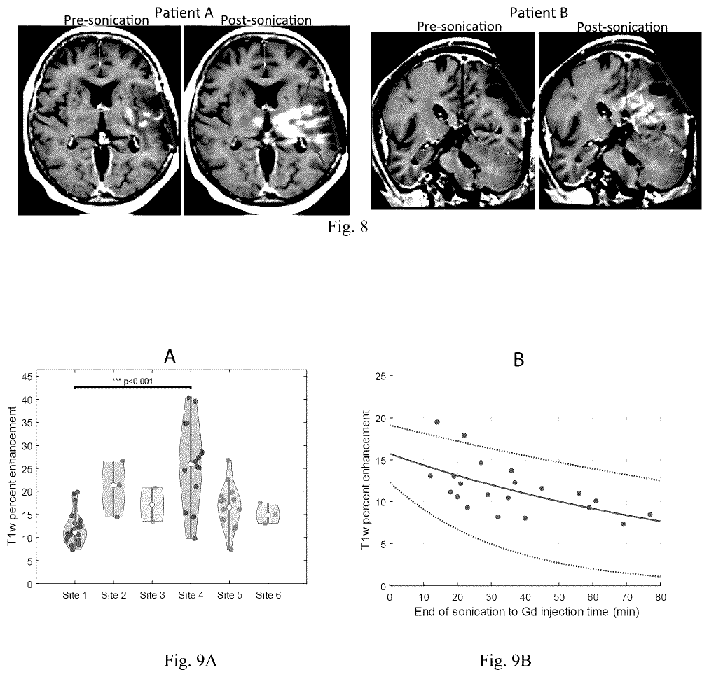

SonoCloud-9 BBB Disruption The extent of BBB disruption was visualized by gadolinium-uptake on T1w MRI performed 24-48 hours before and immediately (within 60 minutes) after sonication (cycle 1 to cycle 3, when performed). Representative images for two different patients are shown in FIG. 8. BBB opening was furthermore evaluated using 61 LIPU/MB procedures in 27 patients in which all nine emitters were activated (Cohorts C, D). Of these 27 patients, additional imaging was performed after cycles 2 and/or 3 providing for a total of 34 additional post-sonication images. A median depth of BBB disruption of 64 mm was observed on T1w-contrast-enhanced imaging and 90% of activated emitters led to grade 2-3 BBB disruption, using the grading criteria previously described (Carpentier et al. 2016, Sci. Transl. Med. 8:343re2; Asquier, et al. 2019, J. Neurosurg. 132:875-883). No difference between first and subsequent sonications were observed. Absence of clear BBB disruption (grades of 0 or 1) was explained by the limitation of the automatic method to analyze poor quality images, and regions with a large volume of resection cavity or tumor enhancement that confounded the algorithm.

Significant differences in extent of BBB disruption were observed between participating investigational sites while results across patients treated at the same center were comparable. The semi-quantitative method to evaluate BBB-disruption intensity based on post sonication MRI is dependent on acquisition parameters: sequence, scanner and contrast agent used. As shown in FIG. 9A, the enhancement intensity was significantly higher at site 004 where Gadovist® was used than in site 001 (p<0.001), where Dotarem® was used. This greater enhancement intensity in the brain of some contrast agents has been reported previously (Anzalone et al. 2013, Eur. J. Radiol. 82:139-145).

Depending on availability of the MRI after sonication, the time between sonication and administration of the gadolinium bolus varied from 10-77 min range (mean: 33±14 min) depending on the immediate availability of the MRI scanner. As shown in FIG. 9B, a significant negative correlation was found between enhancement intensity and sonication to gadolinium time for treatments performed with sufficient data (site 001, analysis on 31 sonications, Spearman correlation, rho=−0.6, p=0.005). An exponential decay fit indicated a half-closure time of LIPU-disrupted BBB of 1.3 hours (95% confidence interval: 0.4-2.2 h).

### Optimized Administration of Carboplatin Led to Improved Radiological Response

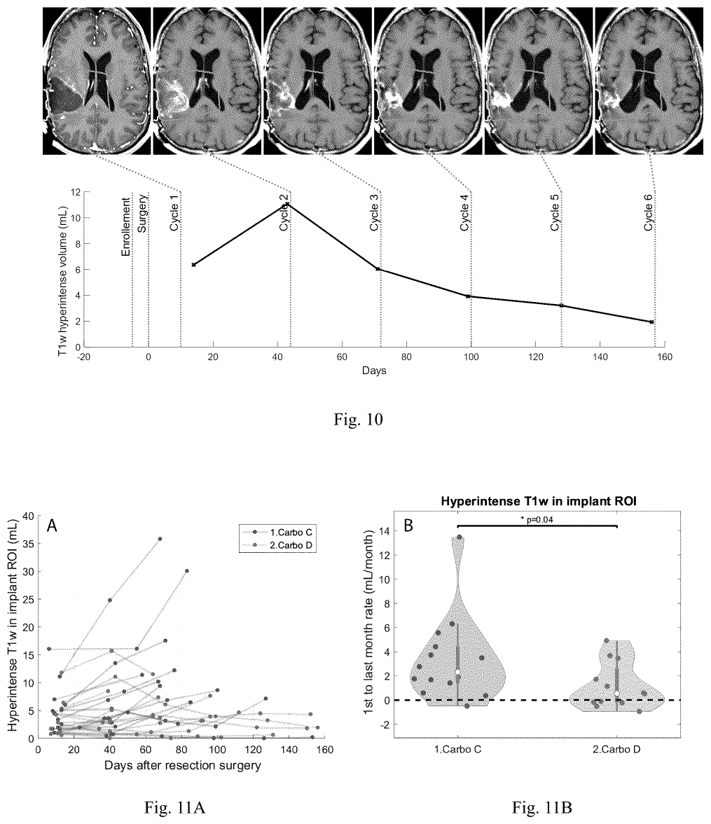

Cohort D evaluated a change in treatment sequence, i.e. sonication to follow immediately after the end of carboplatin infusion and thus also shortening of the interval between chemotherapy administration and sonication to a few minutes only (mean interval between sonication and chemotherapy 64 min versus 14 min in cohorts C and D, respectively). Better in-field tumor control was observed in patients in cohort D compared to cohort C (chemotherapy administration up to 60 minutes after sonication). An example of the T1w contrast-enhanced evolution of the tumor volume from six monthly pre-sonication images is shown in FIG. 10. This patient had an increase in T1 enhancement due to tumor up to Cycle 2 that decreased over time during the monthly treatments.

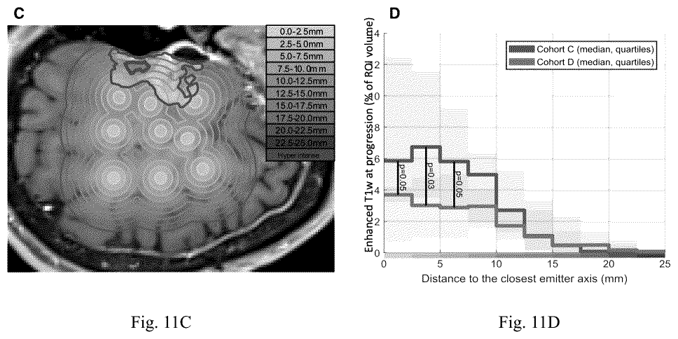

The evolution of the tumor-related hyperintense T1w volume in the region targeted by the implant (shown in green in FIG. 10) was evaluated and is shown in FIG. 11A for 26/27 patients treated with nine emitters in Cohorts C and D. One patient in Cohort C was excluded as they left the study after cycle 1 due to an adverse event (not tumor progression) and additional MRIs beyond pre-cycle 1 were not available. As shown in FIG. 11B, a significantly lower tumor growth rate over the study duration was found in cohort D (median=0.54 mL/month), than in cohort C (median=2.31 mL/month) (Wilcoxon-Mann-Whitney test: p=0.04). When the region targeted by the implant was excluded from the analysis, and only regions outside the sonication field were used, there was no significant difference between the evolution of the T1w enhancement between cohorts C and D (p=0.55).

In order to further evaluate the effect of the treatment, an analysis was performed using the end-of-study MR images from the patients in Cohorts C and D. The contrast-enhancing tumor at the end-of-study was segmented with a semi-automatic method in the post-gadolinium T1w images. The percentages of brain volume comprised between two given distances to emitters axes (tube-shaped regions) covered by tumor mask were computed. An analysis from a representative patient of this analysis is shown in FIG. 11C, in which the nine emitters and distances from each emitter axis are shown along with the region of enhancing tumor volume from the end of study images (red contour). FIG. 11D shows a comparison of the local tumor progression probability metric between patients from Cohorts C and D using this analysis method. There was less likelihood for there to be tumor in the cylindrical zone in front of each emitter for cohort D than for cohort C, up to a cylinder with a radius of 10 mm (with statistical significance up to 7.5 mm, Wilcoxon rank-sum test). Further away from the emitters, no difference between the two cohorts was observed.

### Clinical Outcomes

The median overall survival of patients treated with all nine emitters activated was 12.4 months (95% CI (8.4, 14.0) and compares favorably with other reports of therapies for recurrent GBM. The results are shown below in Table 3.

TABLE 3
Clinical Outcomes of patients with 9 emitters activated (cohorts C and D).

| | mPFS | | | mOS |
|---|---|---|---|---|
| Cohort | (months) | 9-month OS | 1-yr OS | (months) |
| C + D | 2.7 | 70% | 52% | 12.4 |
| (N = 27) | | | | |
| C | 2.5 | 67% | 47% | 11.8 |
| carboplatin after US | | | | |
| (N = 15) | | | | |
| D | 3.1 | 75% | 58% | 14.0 |
| carboplatin before US | | | | |
| (N = 12) | | | | |

In the subset of patients of the cohort D, results indicate possibly improved clinical outcomes compared to patients treated in Cohort C (Table 3) with a slightly longer mPFS (3.1 months [95% CI: 2.14-5.03] vs (2.5 months, 95% CI: 2.14-2.83], p=0.55). The 1-year OS rate was longer in Cohort D (58.3%; [95% CI: 0.15, 0.86]) vs Cohort C (46.7%; [95% CI: 0.09, 0.78]), even though more tumors in cohort D were MGMT unmethylated, thus suggesting that the sequence and or timing of sonication are important factors to allow for adequate drug penetration across the BBB. The median OS was also longer in Cohort D (mOS=14.0 months; [95% CI: 6.70, 17.3]) in comparison to Cohort C (mOS=11.8 months; [95% CI: 8.02, 13.2]) leading to the conclusion that a closer time of administration of carboplatin to sonications led to improved radiological and clinical outcomes.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for delivering a therapeutic agent to a human brain, the method comprising:
delivering systemically a first therapeutic agent to a human, and
applying low intensity pulsed ultrasound (LIPU) in the presence of circulating microbubbles to a brain of the human for a period of 1 μs to 20 min,
wherein the LIPU results in an opening of a blood-brain barrier volume of 1-1500 cm³, the first therapeutic agent is delivered prior to applying the LIPU if a half-life in plasma of the first therapeutic agent is ≥1 h, or the first therapeutic agent is delivered after applying the LIPU if the half-life in plasma of the first therapeutic agent is <1 h.

2. The method of claim 1, wherein a time period between completion of the delivering step and the applying step is not more than 45 min.

3. The method of claim 1, wherein the LIPU is applied within 0 to 30 min after completion of delivery of the first therapeutic agent having a half-life in plasma of ≥1 h.

4. The method of claim 1, wherein the first therapeutic agent having a half-life in plasma of <1 h is delivered over a period of time starting 0 to 30 min after applying the LIPU.

5. The method of claim 4, wherein the LIPU is applied to the brain at a pressure level of 0.5 MPa or higher at a resonance frequency ranging from 0.5 to 1.5 MHz.

6. The method of claim 5, wherein the ultrasound pressure is between 0.5 MPa and 2.00 MPa.

7. The method of claim 1, wherein the LIPU has a mechanical index (MI) from 0.1 to 2.00.

8. The method of claim 7, wherein the LIPU is applied in pulses of duration about 25 ms and a pulse repetition frequency of about 0.5 Hz to 1 Hz.

9. The method of claim 1, wherein the LIPU is applied by an ultrasound transducer implanted in a burr hole or bone flap in a skull of the human.

10. The method of claim 1, wherein the first therapeutic agent is carboplatin, a therapeutic antibody, a CAR T cell, an NK cell, temozolomide, lomustine, carmustine, irinotecan, or topotecan.

11. The method of claim 10, wherein the first therapeutic agent is delivered by infusion over a period not exceeding 60 min.

12. The method of claim 10, further comprising delivering a second therapeutic agent different from the first therapeutic agent.

13. A method for delivering therapeutic agents to a human brain, the method comprising:
delivering systemically a first therapeutic agent to a human,
applying low intensity pulsed ultrasound (LIPU) in the presence of circulating microbubbles to a brain of the human for a period of 1 μs to 20 min, and
delivering systemically a second therapeutic agent to the human,
wherein the LIPU results in an opening of a blood-brain barrier volume of 1-1500 cm³, the first therapeutic agent has a longer alpha half-life than the second therapeutic agent, the first therapeutic agent is delivered prior to the applying step, and the second therapeutic agent is delivered after the applying step.

14. A method for treating a brain disorder in a human, the method comprising:
delivering a first therapeutic agent systemically to a human suffering from a brain disorder,
administering to the human a microbubble solution, and
applying low intensity pulsed ultrasound (LIPU) to a brain of the human for a period of 1 min to 30 min,
wherein the LIPU results in an opening of a blood-brain barrier volume of 1-1500 cm³ and the first therapeutic agent is delivered by infusion starting at 15 to 90 min prior to applying the LIPU.

15. The method of claim 14, wherein the brain disorder is cancer and the first therapeutic agent is carboplatin delivered at a dose of AUC3-7.

16. The method of claim 14, wherein the LIPU is applied for 3 to 5 min.

17. The method of claim 14, wherein the LIPU is applied by an ultrasound transducer implanted in a burr hole or bone flap in a skull of the human.

18. The method of claim 14, wherein the first therapeutic agent is infused through a catheter for a period of 60 min. or less.

19. The method of claim 18, further comprising flushing the catheter with saline for a period of 20 s to 10 min, preparing the microbubble solution, connecting the ultrasound transducer electrically to a radio frequency generator, and injecting the microbubble solution into the catheter, wherein the carboplatin is delivered by infusion for 30 min through the catheter prior to the flushing step and the LIPU is applied for a period of 4.5 min after injection of the ultrasound contrast agent.

20. The method of claim 19, further comprising obtaining a magnetic resonance image of the brain of the human to determine an extent of blood-brain barrier volume opening.
